(19) United States
(12) Patent Application Publication
(10) Pub. No.: US 2017/0007141 A1
(43) Pub. Date: Jan. 12, 2017

(54) SMART WIRELESS ELECTRODE ARRAY

(71) Applicants: Peter Andris Raudzens, Scottsdale, AZ (US); Valdis Victor Rundans, Victoria (CA)

(72) Inventors: Peter Andris Raudzens, Scottsdale, AZ (US); Valdis Victor Rundans, Victoria (CA)

(21) Appl. No.: 14/795,294

(22) Filed: Jul. 9, 2015

(51) Int. Cl. A61B 5/042; A61B 5/04; A61B 5/00

(52) U.S. Cl. CPC A61B 5/0422; A61B 5/0006; A61B 5/04012; A61B 5/6898; A61B 5/0022; A61B 5/0024

(57) ABSTRACT

This invention relates to at least one electrode for subdermal penetration to provide a substantially constant electrode impedance, said electrode associated with a power module and wireless module for receiving and transmitting physiological signals to a processing unit communicating with a server.

### FIELD OF INVENTION

This invention relates to medical information technology, physiological sensing, signal conditioning and display. More particularly this invention relates to an electrode with power and wireless communications modules for receiving arid transmitting signals to a tablet communicating with a cloud server, and in one embodiment relates to an electrode array for contacting skin and with power and Bluetooth (trademark) communications modules.

### BACKGROUND OF THE INVENTION

The recent migration of cloud-based servers and Bluetooth near-field data transfer to social media and information management has exploded. This technology can also be applied for medical information at orders of magnitude greater than current systems to improve signal resolution and to reduce recording costs.

Traditionally physiological signals from any neural generator have been captured, conditioned, amplified, filtered, and displayed from either surface (disk) or needle electrodes.

Disc electrodes are compromised by inconsistent impedance values due to changes in skin, hair, oil etc resistance between the contact surface and the neuroelectric generator. Needle electrodes are invasive and complicate the test with the risk of hematoma or infection The noisy signal picked up by prior art disc or needle electrodes is transmitted by dedicated wires to a hard-wired processor for signal identification. However there are interference/impedance problems created by the wires and traditional electrodes that need to be filtered and dealt with so as to clean up the signals so they can be effectively used. The algorithms for various physiological signals have been refined with clever features such as rate limiting and pattern recognition, but the basic problems of antenna-induced noise and mechanical nuisance remain in any wired medical monitoring system.

These problems have not been corrected by conventional medical device manufacturers for more than 30 years because there is no economic incentive to retool if there is no competitive model of a better and cheaper way to capture physiological data.

Prior art devices and methods include for example U.S. Pat. No. 7,060,031 relates to a method and apparatus for remotely programming implantable, medical devices. The apparatus includes a server adapted to receive and store at least one request to modify the behaviour of an implantable medical device provided by a programmer adapted to allow a clinician to create at least one request at a first selected time. The apparatus further includes a monitor adapted to receive the requests from the server and transmit the requests to the implantable medical device at a second selected time and a bi-directional communications system adapted to couple the server and the monitor.

Also U.S. Pat. No. 8,708,903 relates to a wireless system for a person which includes a wearable appliance monitoring one or more body parameters; a plurality of wireless nodes in communication with the wearable appliance; and a remote computer coupled to the wireless nodes to provide information to an authorized remote user.

Moreover U.S. Pat. No. 8,839,319 teaches an ECG monitoring system for ambulatory patients includes a small multi-electrode patch that adhesively attaches to the chest of a patient. A reusable battery-powered ECG monitor clips onto the patch and receives patient electrical signals from the electrodes of the patch. A processor continuously processes received ECG signals and stores the signals in memory in the monitor. Processed ECG signals and cardiac event information are sent wirelessly to a cellphone handset for relay to a monitoring center The ECG monitor is contained in a watertight sealed case with only electrical contacts or the outside of the case. The electrical contacts electrically couple the ECG monitor to the electrodes of the patch during patient monitoring and to a charger during recharge of the battery.

Also U.S. Pat. No. 8,907,782 relates to a wireless medical device comprises a processor, a memory, a sensor for detecting a physiological signal, a radio and a proximity detector to measure a distance of the wireless medical device relative to a second wireless medical device. The proximity detector includes a ranging functionality. A wireless communication channel is created when a distance between the wireless medical device and the second wireless medical device is within a first predetermined threshold, The distance is greater than zero.

Furthermore US publication 20130281815 shows a system for cardiac monitoring of an individual. The system includes a garment having a plurality of nanostructured textile electrodes integrated therein, the electrodes arranged on the garment to record data for an EGG of the individual; a first controller electrically coupled to the plurality of electrodes, the controller including a wireless transmitter, the first controller being configured to collect the recorded data for the ECG from the plurality of electrodes and to cause the wireless transmitter to wirelessly transmit the recorded data; and a wireless receiving station including a wireless receiver and a second controller, the second controller configured to cause the wireless receiver to receive the recorded data transmitted by the wireless transmitter, analyze the recorded data for the EGG, analyze the recorded data, identify an abnormality in the ECG, and generate an alert if an abnormality in the ECG is identified.

Finally U.S. Pat. No. 3,943,918 relates to a throwaway, one-time use signal sensing and telemetric transmitting device for use such as in the care of medical patients requiring a monitoring of a physiological function such as the cardiac function of the patient. The device includes one-time use self-powering battery means, adhesive means for attachment of the device to the patient and electrodes for sensing the physiological functioning. A disposable cover is removed to expose the adhesive means and the battery means are actuated to power the device at the time of use. The radio frequency transmitted signal is received on a suitable radio telemetry receiver for monitoring and recording as desired. However this patent uses disc type electrodes and old radio technology

There is a need for an improved electrode, method and system incorporating the electrode, that is simple to manufacture, and use as compared with, the prior art.

It is an aspect of this invention to provide an electrode for subdermal penetration to provide a substantially constant electrode impedance, said electrode associated with a power module and wireless module for receiving and transmitting physiological signals to a processing unit communicating with a server.

It is another aspect of this invention to provide an electrode array for subdermal penetration to provide a substantially constant electrode impedance, said electrode array having a power module and Bluetooth (trademark) relays for receiving and transmitting physiological signals to a tablet communicating with a cloud server.

It is still another aspect of this invention to provide a system for monitoring physiological signals such as EGG signals of a patient wearing an electrode array where the electrode array subdermally penetrates the skin of a patient to provide a substantially constant electrode impedance: a power unit; a wireless communication unit associated with said electrode array for communicating with a tablet; said tablet receiving and transmitting signals from said electrode array and from a cloud server.

These and other objects and features of the invention shall now be described in relation to the following drawings.

### DESCRIPTION OF THE DRAWINGS

FIG. 1 is a general schematic of the system.

FIG. 2 is a representative view of an electrode.

FIG. 3 is a representative view of the components of the invention.

FIG. 4 is a view of one embodiment of the invention.

FIG. 5a is a cross sectional view of FIG. 4.

FIG. 5b is a cross sectional view of another embodiment of the invention where the layers have been switched.

FIG. 6 is a representative view of another embodiment of the invention.

FIG. 7 is an example of use of the components of FIG. 6.

### SUMMARY OF THE INVENTION

The Smart Wireless Electrode Array is the basic building block of a better model for medical information technology. In one embodiment this invention relates to a multipurpose wireless electrode that can capture an array of physiological signals. The system is scalable and can be tailored to individual test requirements.

In one embodiment the invention relates to the acquisition and display of a electrocardiogram (EKG) signal. For example the recording electrode is a 2″×6″ flexible mesh array of 6 tapered metallic contacts embedded in an inert porous membrane with an adhesive film surface wired to a Bluetooth transmitter (see diagram). Each electrode will penetrate the epidermal skin layer to a depth of approximately 1 mm. This will provide constant inter-electrode impedance without the complications of needle electrodes. More particularly by subdermally “biting” the patient, a constant impedance is achieved which allows the acquisition of a clearer signal which does not require as much filtering and manipulation as do existing electrode systems.

This electrode array can foe shaped and configured for any specific application. It is sterile, disposable, and non-toxic.

The electrical activity recorded by the array of tapered electrodes is transmitted by the Bluetooth device through a receiver in any inexpensive tablet 6 to both condition and display the signal. The display will utilize alarm limits and pattern recognition paradigms. The signal conditioning and display algorithms can be migrated from cloud based servers or some hybrid combination of dedicated tablet chips.

An initial ECG wireless model is the basis for a variety of medical applications at lower cost and better resolution. This design is the first stage of a planned medical product. Initially each of 4 remote electrodes will be wired separately to individual transmitters (Bluetooth, Wi-Fi or Zig Bee). This arrangement will identify the characteristic changes of voltage separated spatially arid temporally from each active electrode as shown in the figures.

The corresponding receivers are incorporated in a tablet 6 that can be separated by a distance of 150 feet, for example. The tablet 6 functions to both condition and display the signal. Conditioning involves analog-digital conversion, amplification, filtering, and signal formatting. Signal display involves appropriate scaling in a time/power graph. Other features such as rate limit alarms and pattern recognition modules will be added.

The next step in the design is to integrate 4 to 6 channels of physiological data into a single transmitter and simplify the wireless device. This step is independent of the signal conditioning and display application created on the dedicated tablet.

The Cross-Sectional views 5a and 5b clearly defines the features of this Smart Wireless Electrode Array; a detachable and reusable communications and power modules layer, an impervious waterproofing layer, an electrode and micro-wiring layer, a porous flexible sterile inert membrane layer with an adhesive film on the skin side of the patient. The three layers below the top layer are detachable and discardable. The improved electrode design consists of pyramidal tapered metallic electrodes with points that penetrate sub-dermally to a depth of 1 mm.

The point of this Smart Wireless Electrode Array is not only the reduction of clutter and the wire nuisance around the patient, but the provision of a cleaner, less noisy physiological signal which requires less conditioning for display and use by medical personnel. In addition, obsolete technology in the form of 30 year-old “black box” technology can be eliminated just as personal computers have been made obsolete through the use of tablets and cloud computing.

### DETAILED DESCRIPTION OF THE INVENTION

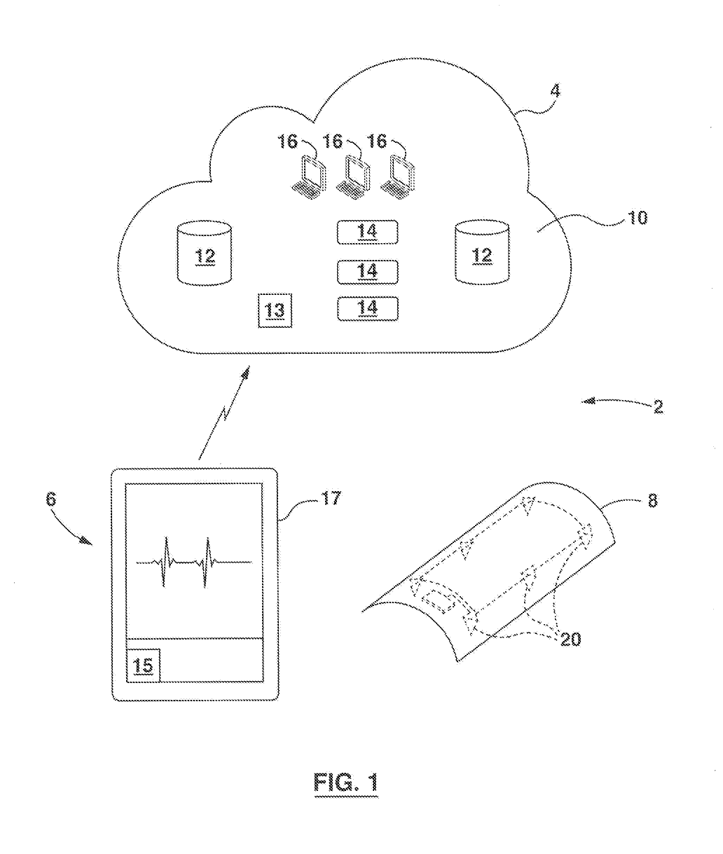

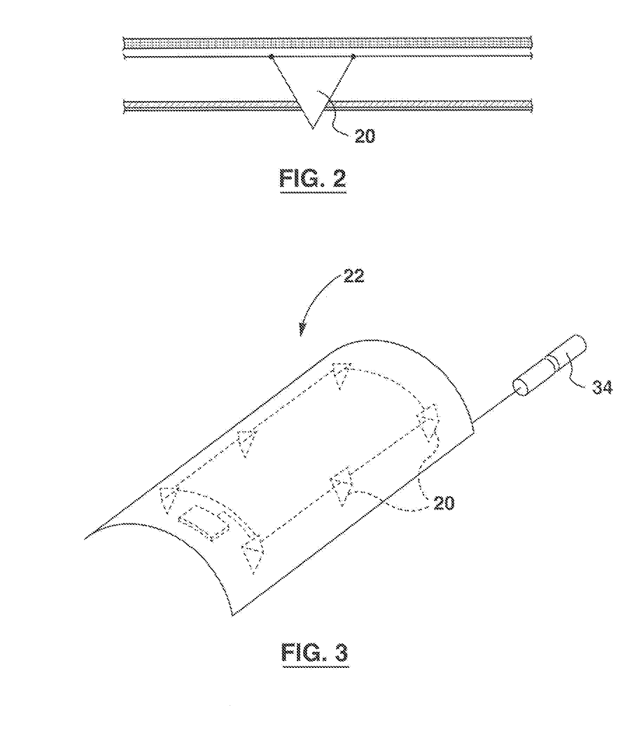

FIG. 1 generally illustrates the system 2 which comprises the cloud 4, healthcare professional computing device 6 and electrode means 8. Generally speaking the cloud 4 or cloud computing consists of storing and accessing data and programs over the internet 10 rather than ones hard drive. Data is stored in a data base 12 and programs may be stored in application servers 14. The cloud may also include computer networks 16 connected to the internet that are connected to the application servers 14 and data base 12.

Advantages of utilizing the system 2 and particularly the cloud 4 include the ability to utilize and run different software programs on the various application servers 14. So for example an electrocardiogram facility or program can be stored on one of the application servers 14 while a blood oxygen facility may be stored on another application software server 14 for monitoring and control and so on. Up to seven slave sensors are proposed: Temperature, Blood Pressure, EKG, O2, CO2, EEG, and EP Evoked Potentials. Blood Sugar sensing is specifically excluded. Updates to the programs can be centralized in the cloud and updates to the actual hardware comprising the computer networks 14 can be managed and purchased by others so as to free up the medical profession to concentrate on what they do best, and that is to diagnose and treat patients. Furthermore the medical space become free of clutter as the wires, connectors, hardware and software is located elsewhere in the “cloud”. Also the data that is stored in the data base 12 in the cloud 4 can be accessed from any authorized computer 6 from any location, making it easier for the healthcare professional to access.

It should be noted that in another embodiment the system 2 could also work by storing the selected software application in a computer or server 14a located at the medical institution or doctors office hut connected to the internet 10 so long as the healthcare professional computing device 6 used by the healthcare professional is capable of communicating with the computer or server 14a and a data base 12a that may be in the cloud 4 or associated with the computer or server 14a.

The healthcare professional computing device 6 can consist of tablets, lap tops, desk top computers with screens, personal digital devices such as smart phones or the like.

The electrode means 8 is schematically disclosed in FIG. 1 and can consist of at least one electrode 20. In one embodiment the electrode has a conical or pyramidal shape that will pierce or penetrate the epidermal skin layer to a about a depth of 1 mm. This will provide constant inter-electrode impedance without the complications of needle electrodes.

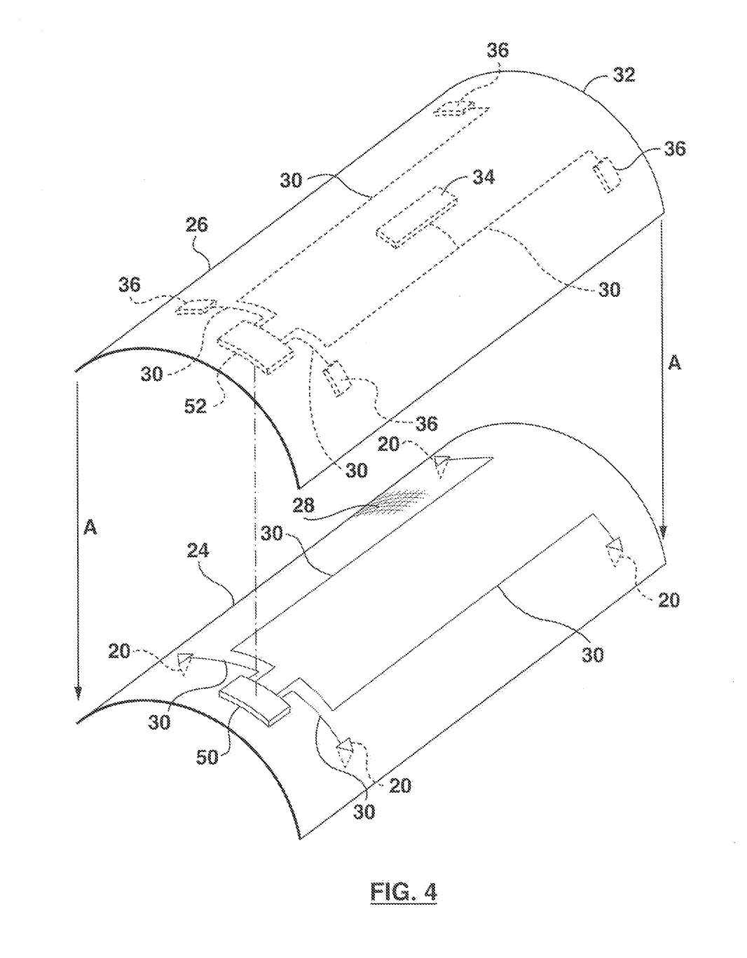

In another embodiment the electrode means can: comprise an electrode array 22 that is shaped and configured for any specific application as generally shown in FIG. 3. It is sterile, disposable, and non-toxic. The mesh 28 can be cut to any configuration, including shaping the mesh 28 into a cap to be worn by a patient for capturing brain signals or the like. FIG. 3 also shows a battery connection 34 to power the electrode array 22.

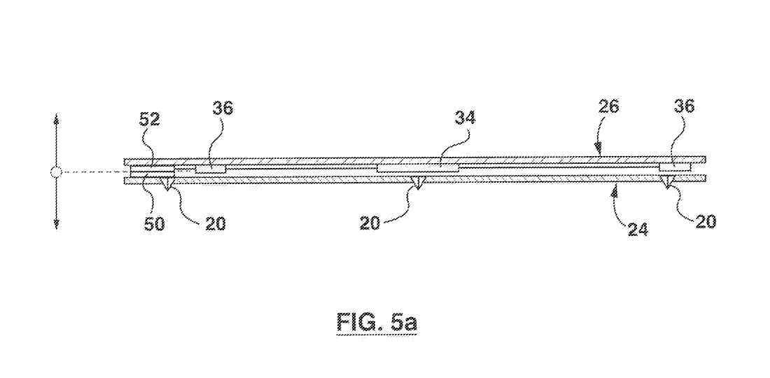

More particularly FIG. 4 illustrates one application of the electrode array 22 that comprises a first component 24 and a second component 24. As shown in FIG. 4 the first component 24 is a lower component and the second component is the upper component 26. The lower component 24 includes the electrodes 20 that are embedded info a suitable material such as a mesh 28. The electrodes are electrically connected by conductors 30. The upper component 26 can include a power source 34 which can consist of a battery or rechargeable battery or other power means 34. The upper component also includes wireless units 36 that are connected to the power source 34. In one embodiment the wireless units 36 consist of Bluetooth (trademark) units or relays are capable of sending or receiving signals Other sending and receiving -units can be used within the scope of this invention; however Bluetooth (trademark) is relatively safe: stable and more secure than other technologies. The upper and lower components are electrically connected together by connectors 52 and 50 respectively when they contact each other.

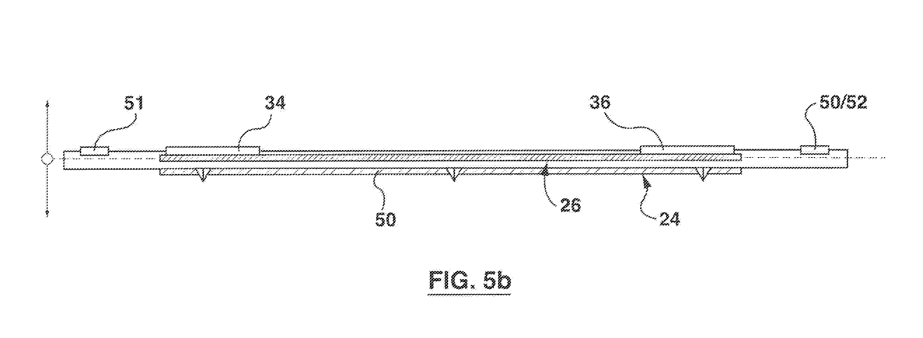

FIG. 5a is a cross-sectional view that illustrates the components of FIG. 4 when they come together so as to contact each other as shown by arrows A in FIG. 4.

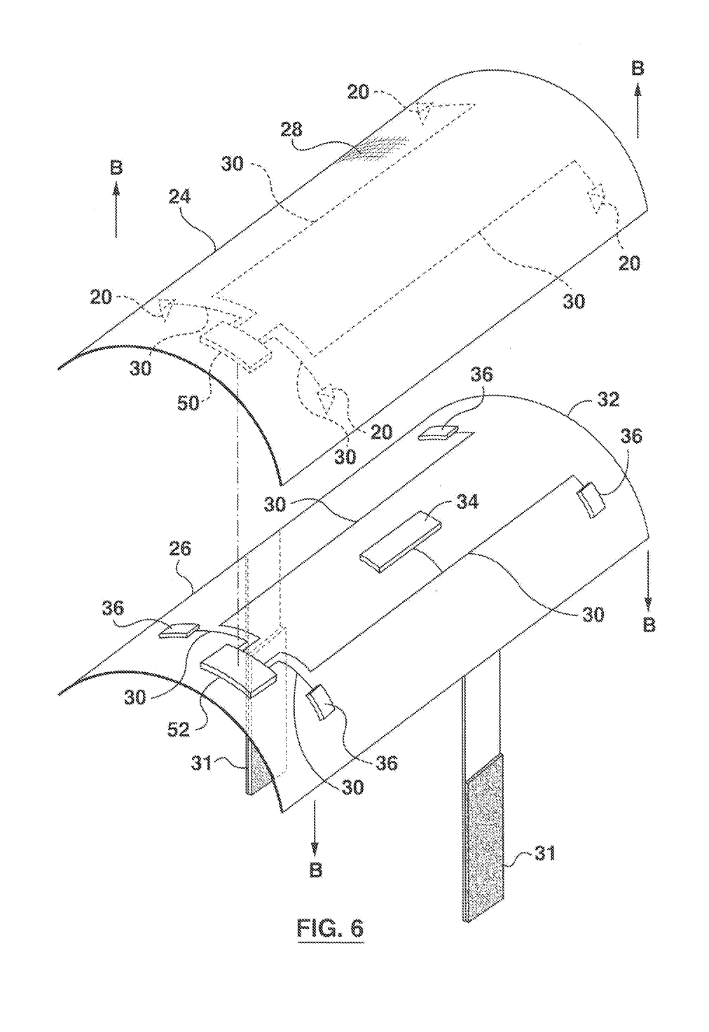

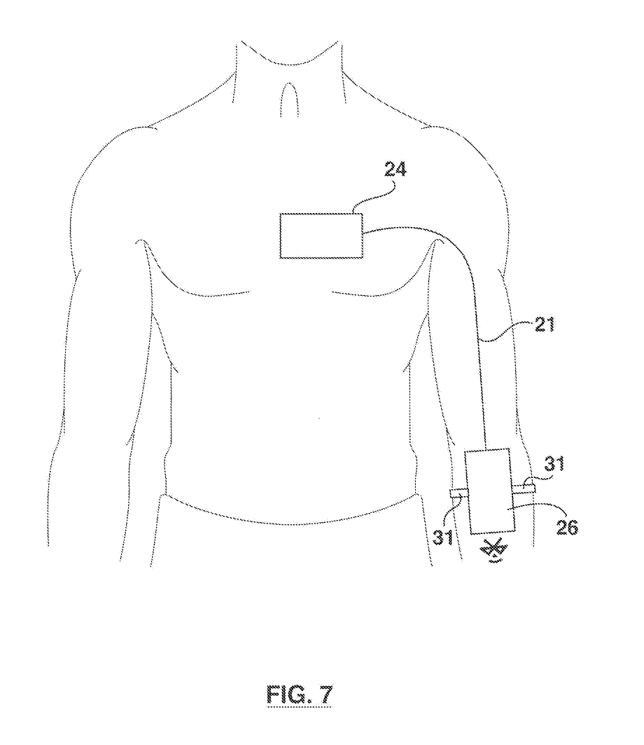

FIG. 6 shows another embodiment where the first and second components are reversed in relation to FIG. 4. In other words the upper component 24 in FIG. 6 consists of a removable portion 32 that can he connected to the patient such as the patient's chest as shown in FIG. 7 and the second or lower component in FIG. 6 can be connected to another part of the body such as the forearm of the patient straps 31 having loop pile and hooks such as sold under the trademark Velcro as detailed in FIG. 7. The straps 31 are adjustable to fit the patient. The portion 32 can include a power source 34 which can consist of a battery or rechargeable battery or other power means 34. The portion 32 also includes wireless units 36 that are connected to the power source 34. The electrodes 20 and conductors or wiring harness 30 are carried by the first component 24. In one embodiment the wireless units 36 consist of Bluetooth (trademark) units or relays are capable of sending or receiving signals. Other sending and receiving units can be used within the scope of this invention; however Bluetooth (trademark) is relatively safe, stable and more secure than other technologies.

In one embodiment FIG. 7 also shows an electrical connector connecting first and second components 24 and 26. This corresponds to the embodiment shown in FIG. 6. The preferred embodiment is shown in FIGS. 4, 5a or FIG. 5b.

Alternatively one Bluetooth (trademark) unit 36 can be used with a plurality of sampling rates to identify one of four separate recording electrodes 20.

The upper and tower components 24 and 26 are electrically connected through releasable connectors 50 and 52.

FIG. 5b is a cross sectional view through another embodiment of the attachment of the first and second components 24 and 26. In other words the first component or layer 24 in FIG. 5a show the electrodes 20 in the bottom or lower layer 24 as does FIG. 5b. Second layer 26 includes the power source or module 34 and communication modules or Bluetooth (trademark) units 36. FIG. 5b also shows a power module connector 51 and connectors 50/52. The electrodes 20 in FIG. 5b are disposed in the lower component or layer 24. The electrodes 20 in one embodiment comprise pyramidal tapered metallic electrodes.

The lower layer 26 can consists of a porous flexible sterile inert membrane 50 with an adhesive film membrane film on the skin side. A waterproof layer 52 separates the first and second layers 24 and 26.

In one embodiment of the invention the first layer 24 is detachable from the patient and can be discarded while the second layer 26 can he detached and reused.

FIG. 1 is a representative drawing showing the system 2 in use where a tablet 6 can be activated so as to communicate by means of a Bluetooth (trademark) unit or relay 15 to activate the desired application from an application server 14. For example if a healthcare professional desires to conduct an electrocardiogram (EGG) of a patient the healthcare professional will select the appropriate application from the tablet 6 that will communicate with a control node 13 for authorized connection to the appropriate program (ie the ECG program or app) from the cloud so as to enable the tablet 6 to display the signal on the display 17.

The signal conditioning, analogue to digital conversion (A/D) filtering and amplification can take place in the cloud through the use of algorithms, making it unnecessary to clutter the heath care facilities with these devices.

Once the desired app through the tablet 6, is selected, the tablet 6 then communicates with electrode means as previously described to display the ECG signal and to capture this data in the data base 12 for future retrieval from the cloud as previously discussed.

1. An electrode for subdermal penetration to provide a substantially constant electrode impedance, said electrode associated with a power module and wireless module for receiving and transmitting physiological signals to a processing unit communicating with a server.

2. A electrode as claimed in claim 1 wherein said processing unit comprises a tablet.

3. A electrode as claimed in claim 2 wherein said server comprises a cloud server further including a data base, application servers and computer networks.

4. An electrode as claimed in claim 3 wherein said electrode comprises an electrode array.

5. An electrode array for subdermal penetration to provide a substantially constant electrode impedance, said electrode array having a power module and Bluetooth (trademark) relays for receiving and transmitting physiological signals to a tablet communicating with a cloud server.

6. An electrode array wherein said signal is an ECG signal.

7. A system for monitoring physiological signals such as ECG signals of a patient wearing an electrode array where the electrode array subdermally penetrates the skin of a patient to provide a substantially constant electrode impedance: a power unit; a wireless communication unit associated with said electrode array for communicating with a tablet; said tablet receiving and transmitting signals from said electrode array and from a cloud server.

8. A system as claimed in claim 7 wherein said electrode array has an upper layer and lower layer.

9. A system as claimed in claim 8 wherein one of said upper or lower layers includes at least one electrode penetrating the skin of a patient to provide a substantially constant electrode impedance.
